(19) United States
(12) Patent Application Publication
(10) Pub. No.: US 2003/0171333 A1
(43) Pub. Date: Sep. 11, 2003

(54) Pharmaceutical composition containing at least a polymer combined with or conjugated to at least a phenylalkylcarboxylic acid salt, conjugate polymers and uses thereof

(76) Inventors: Thierry Avramoglou, Deuil-La Barre (FR); Rosita Bagheri, Pearland, TX (US); Frederic Chaubet, Eaubonne (FR); Michel Crepin, Paris (FR); Latifa Dahri, St Amand Les Eaux (FR); Melanie Dibenedetto, Gagny (FR); Claudia Gervelas, Paris (FR); Remi Huynh, St Amand Les Eaux (FR); Jacqueline Jozefonvicz, Lamorlaye (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/297,035

(22) PCT Filed: May 30, 2001
(86) PCT No.: PCT/FR01/01672

(30) Foreign Application Priority Data
Jun. 2, 2000 (FR) 00/07117

(51) Int. Cl. A61K 31/722; A61K 31/721; A61K 31/718; A61K 31/717; A61K 31/716

(52) U.S. Cl. 514/54; 514/55; 514/57; 514/59; 514/60

(57) ABSTRACT

The invention concerns a pharmaceutical composition containing at least a polymer associated or conjugated with at least a phenylalkylcarboxylic acid derivative, novel polymers conjugated with at least a phenylalkylcarboxylic acid derivative and their uses in particular in cancer treatment.

The present invention relates to a pharmaceutical composition comprising at least one polymer combined with or conjugated to at least one phenylalkylcarboxylic acid derivative, to novel polymers conjugated to at least one phenylalkylcarboxylic acid derivative and to uses thereof, in particular in the treatment of cancer.

For the purpose of the present invention, the term “polymer” is intended to mean macromolecules comprising at least one hydroxyl function.

The polymers comprising at least one hydroxyl function, which is in particular the case for poly-alcohols and polysaccharides, may be in their native form or else may be functionalized, given that the hydroxyl function(s) which they comprise can be readily substituted by esterification or etherification.

Polysaccharides are divided up into two classes depending on whether they are made up of molecules of monosaccharides which are all identical (homopolysaccharides), or different (heteropolysaccharides).

Among the homopolysaccharides, mention may first of all be made of glucosans, such as starch, glycogen, cellulose, dextran and chitin. Along with glucosans, mention may also be made of arabans, xylans and pectins.

Among the heteropolysaccharides, mention may in particular be made of gums which are branched structures comprising D-galactose, L-arabinose, L-rhamnose and D-glucuronic acid, and hemicelluloses.

Dextran is a neutral polysaccharide derived from bacteria such as Leuconostoc mesenteroide and Leuconostoc dextranicum. These bacteria have an enzyme, dextransucrase, which transforms sucrose into macro-molecules composed of α(1,6)-linked D-glucose units comprising several hydroxyl functions. The colloidal properties of dextran and also its weak immunogenicity have made it possible to use it as a plasma substitute. Given that the hydroxyl groups of dextran can be readily functionalized, many dextran derivatives have been prepared. Depending on the nature of the substituents and on the degree of substitution, these products have biological properties which are much more extensive than those of native dextran.

In the particular case of functionalized dextrans, it has, for example, been described that certain carboxymethyl benzylamide derivatives of dextran (CMDB), depending on their degree of substitution, exhibit antiproliferative activity on various cell lines, in particular on the HBL100 and HH9 breast pretumor lines and on human breast cancer cells (MCF-7), (R. Baghéri-Yamand et al., 1994, In Vitro Cell. Dev. Biol. 30A, 8822-8824). It has been shown that this antiproliferative activity is associated with the presence of a benzylamide group. The inhibition of cell growth is associated with a decrease in the number of cells in the S phase and with an accumulation of cells in the G₀/G₁ phase (R. Baghéri-Yarmand et al., Anticancer Res., 1992, 12, 1641-1616). Recent studies have shown that certain CMDBs modify the interaction of the various growth factors (FGF 2, FGF 4, PDGF BB, TGFβ) with their membrane-bound receptors (see, for example, P. Bittoun et al., Biochemical Pharmacology, 1999, 57, 1399-1406). CMDBs exercise their antiproliferative activity by interfering with paracrine and autocrine circuits (Liu J F. et al., Anticancer Res., 1997, 17, 253-258; Baghéri-Yarmand R. et al., Biochem. Biophys. Res. Commun., 1997, 239, 424-428; Baghéri-Yarmand R. et al., Br. J. Cancer., 1998, 78, 111-118 and Baghéri-Yarmand R. et al., Cell Growth. Differ., 1998, 9, 497-504). In vivo tests have demonstrated, in athymic (or nude) mice, that the growth of tumors induced by inoculation of an MCF-7ras human breast cancer cell model is blocked by treatment with CMDB. Histological analysis has shown a decrease in the vascularization with these tumors. It has also been shown that certain carboxymethyl benzylamide sulfonate derivatives (CMDBS), depending on their degree of substitution, also have an antiproliferative action on the HBL100 and HH9 cell lines (Baghéri-Yarmand R. et al., Biochem. Biophys. Res. Commun., 1997, 239, 424-428).

Still with the aim of finding anticancer therapies, it has also already been proposed to use certain phenylacetic acid salts, such as, for example, sodium phenylacetate (NaPA). NaPA is a common metabolite of phenylalanine, usually present in the plasma at μmolar concentrations. Higher concentrations of NaPA (of the order of mmolar) lead to cytostasis and reversion of several human cancer cell phenotypes (see in particular Samid D. et al., Cancer Res., 1992, 52, 1988-1992; Samid D. et al., Blood, 1992, 80, 1576-1581; Samid D. et al., J. Clin. Invest., 1993, 91, 2288-2295, and Samid D. et al., Cancer Res., 1994, 54, 891-895). NaPA, and derivatives thereof, also exhibit antitumor activity with respect to the model of MCF-7ras cancer cells implanted in athymic mice (Adam L. et al., Cancer Res., 1995, 55, 5156-5160). NaPA has been used in phase I and in phase II clinical trials, carried out on patients having malignant tumors of the prostate, medulloblastomas or gliomas (Thibault A. et al., Cancer Res., 1994, 54, 1690-1694 and Chang S M. et al., J. Clin; Oncol., 1999, 17, 3, 984-990). The mechanism by which NaPA inhibits cell proliferation is reversible and dependent on the dose administered.

The aim of an anticancer treatment is to kill the tumor cells or to block the proliferation thereof, while at the same time sparing the normal cells. In order to be effective against tumor cells, NaPA must be used, in vivo, at concentrations of the order of 150 mg/kg. However, at this concentration, NaPA have a cytotoxic effect both on the cancer cells and on the healthy cells, which is not desirable.

For this reason, a real need exists to develop novel anticancer treatments which do not have a cytotoxic effect on healthy cells.

It has already been proposed, in particular in U.S. Pat. No. 5,710,178, to combine NaPA with certain substances having a biological activity, such as, for example, interferons, interleukins, tumor necrosis factors, glutamine antagonists, hormones, vitamins, etc. This type of combination does not make it possible to decrease the doses of NaPA used.

The inventors have developed the subject of the invention in order to remedy these problems and to provide a novel anticancer agent.

A subject of the present invention is a pharmaceutical composition, characterized in that it comprises, as active principle, at least one composition chosen from the group consisting of:

a) a conjugate of a polymer comprising at least one free hydroxyl function and of at least one phenylalkylcarboxylic acid derivative of formula (I) below:

R₂—(CH₂)ₙ—COOR₁ (I)

in which:

R₁ represents a hydrogen atom, a halogen atom, an atom of a monovalent alkali metal such as sodium or potassium, or a group —CO(CH₂)ₘR₃,

R₂ and R₃ represent an unsubstituted phenyl radical,

n and m, which are identical, are integers of between 1 and 3 inclusive;

said conjugate corresponding to formula (III) below:

(Polymer)-(OOC—(CH₂)ₙ—R₂)_{d} (III)

in which the polymer comprises at least one free hydroxyl function, n is an integer of between 1 and 3 inclusive, R₂ represents an unsubstituted phenyl radical and d, which is the index of substitution (ds) of derivative of formula (I), is ≧0.05 and ≦1.5, and preferably >0.15, and even more preferentially between 0.5 and 0.8 inclusive;

b) a combination of at least one polymer having a molecular mass of greater than or equal to 5000 Da and comprising at least one free hydroxyl function, and of at least one phenylalkylcarboxylic acid salt of formula (I′) below:

R′₂—(CH₂)_{n′}—COOR′₁ (I′)

in which:

R′₁ is an atom of a monovalent alkali metal such as sodium or potassium,

R′₂ represents an unsubstituted phenyl radical,

n′ is an integer of between 1 and 3 inclusive;

c) and mixtures thereof,

and optionally one or more other additional active principles, and possibly in the presence or not of a pharmaceutically acceptable vehicle and/or of a physio-logically acceptable carrier.

According to the invention, the conjugate compounds of formula (III) above are trivially called NaPACs.

Also according to the invention, the pharmaceutically acceptable vehicle may consist of one or more excipients and/or of one or more transporters such as liposomes.

The polymers used in accordance with the invention may be natural (native) or synthetic, and may be optionally substituted (functionalized), at the free hydroxyl functions, with one or more functional chemical groups chosen from carboxylic; alkylcarboxylic, such as, for example, methylcarboxylic; arylalkylcarboxylic; N-benzylethylenecarboxamide; sulfate; and sulfonate groups, capable of conferring on these polymers additional biological or physicochemical properties.

The polymers which can be used according to the present invention are preferably chosen from natural or synthetic, and optionally functionalized, polyols and polysaccharides.

Among the polyols, mention may in particular be made, by way of example, of polyalkylene glycols, such as polyethylene glycol and polypropylene glycol, and polyvinyl alcohols.

According to an advantageous embodiment of the invention, the polymers are chosen from natural or synthetic, and optionally functionalized, polysaccharides, among which mention may in particular be made of glucosans such as starch, glycogen, celluloses, dextrans, poly-β-1,3-glucans and chitin; arabans; xylans and pectins.

The natural or synthetic, optionally functionalized, dextrans and poly-β-1,3-glucans are particularly preferred, in particular when the pharmaceutical composition in accordance with the invention contains said polymer(s) and the phenylalkylcarboxylic acid salt(s) of formula (I′) in combined form.

By way of functionalized synthetic dextrans, mention may in particular be made of the compounds described in patent applications EP-A-0 023 854; EP-A-0 146 455 or WO 99/29734, such as, for example, dextrans comprising one or more carboxylic, methylcarboxylic, N-benzylmethylenecarboxamide, sulfate and/or sulfonate functions.

Among these functionalized synthetic dextrans, the dextran derivatives of formula (II) below are preferred:

DMCₐB_{b}Su_{c} (II)

in which:

D represents a polysaccharide chain, preferably consisting of sequences of glucoside units,

MC represents methylcarboxylic groups,

B represents N-benzylmethylenecarboxamide groups,

Su represents sulfate groups (sulfatation of the free hydroxyl functions borne by the glucoside units),

a, b and c represent the degree of substitution (ds), respectively of the groups MC, B and Su; a being equal to 0 or ≦2; b being equal to 0 or ≦1; c being equal to 0 or ≦1; it being understood that the sum of a+b+c≦3.

The dextrans of formula (II), and also the method for the preparation thereof, are described in patent application WO 99/29734. These dextran derivatives of formula (II) are trivially called DMCBs and are considered to be copolymers consisting of imaginary R—OX subunits, X possibly being a methylcarboxyl (MC), benzylamide (B) or sulfate group. The polysaccharide chain is therefore considered to consist of 300% of imaginary R—OH subunits, instead of 100% of glucoside units. Thus, a methylcarboxylic dextran (DCM) with a degree of substitution (ds) of methylcarboxylic groups of 1.2 contains 1.2 substituted groups (R-MC) and 1.8 free hydroxyl groups (R-OH), per glucoside unit.

Among the dextran derivatives of formula (II) described above, the compounds in which 0.5≦a≧1.5; 0.1≦b≧1; and 0≦c≧0.6 are preferred.

Among these dextran derivatives of formula (II), preference is given even more particularly to those in which:

i) a=0.70; b=0.3; c=0;

ii) a=0.91; b=0.18; c=0;

iii) a=0.67; b=0.33; c=0; (named LS4 DMCB),

iv) a=0.68; b=c=0; (named LS12),

v) a=0.67; b=0.39; c=0; (named LS17),

vi) a=0.68; b=0.34; c=0;

vii) a=0.68; b=0.20; c=0;

viii) a=0.7; b=0.3; c=0; (named DMCB7),

ix) a=0.60; b=0.40; c=0; (named LS17-DMCB),

x) a=0.60; b=0.35; c=0; (named DMCB10).

Among the phenylalkylcarboxylic acid salts of formula (I′) above, mention may in particular be made of sodium phenylacetate, potassium phenylacetate, sodium phenylpropionate, potassium phenylpropionate, sodium phenylbutyrate and potassium phenylbutyrate.

When the pharmaceutical composition in accordance with the invention contains at least one dextran of formula (II) conjugated to at least one compound of formula (I), the conjugate compound thus formed corresponds to the following formula (IIIa):

(DMCₐB_{b}Su_{c}).(OOC—(CH₂)ₙ—R₂)_{d} (IIIa)

in which D; MC; C; B; Su; R₂; a, b, and c have the same meanings as those given above for the compounds of formulae (I) and (II) and d has the same meaning as that given above for the compounds of formula (III), the sum a+b+c+d being less than or equal to 3.

According to a particularly preferred embodiment, the pharmaceutical composition in accordance with the invention contains at least one conjugate compound of formula (III) described above, and even more preferentially at least one conjugate compound of formula (IIIa).

Among the conjugate compounds of formula (IIIa) above, preference is given to those in which a>0.5; b>0.15; c=0 or <0.8 and d<1.5.

Even more particularly, preference is given to the conjugate compounds of formula (IIIa) in which:

i) a=0.68; b=0.20; c=0 and d=0.14;

ii) a=0.64; b=0.43; c=0 and d=0.35;

iii) a=0.67; b=0.39; c=0 and d=0.35 (LS17-NaPAC);

iv) a=1; b=c=0 and d=0.1; and

v) a=1; b=c=0 and d=0.3.

The polymer(s) combined with at least one phenylalkylcarboxylic acid salt of formula (I′) and the conjugate compounds of formula (III) or (IIIa) surprisingly have an anticancer activity greater than that of each of the compounds used separately, consequently making it possible to decrease the effective doses of each of the active principles, while at the same time increasing the anticancer efficacy of the pharmaceutical composition containing them.

Also surprisingly, this anticancer activity is even greater when a conjugate compound of formula (III) or (IIIa) is used.

When the polymer(s) and the phenylcarboxylic acid salt(s) of formula (I′) are present in the pharmaceutical composition in accordance with the invention in the combined form, then the ratio of concentrations (expressed in mM) between the polymer(s), firstly, and the compound(s) of formula (I′), secondly, is preferably between 10:1 and 1:1, and even more preferentially between 10:2 and 1:1.

The pharmaceutical composition in accordance with the invention may, of course, contain one or more conventional additives used for preparing pharmaceutical compositions, such as antiaggregating agents, antioxidants, dyes, flavor modifiers, or smoothing, assembling or isolating agents, and, in general, any excipient conventionally used in the pharmaceutical industry.

The pharmaceutical compositions in accordance with the present invention have a certain number of biological activities: antiproliferative, cytostatic, necrotizing, proapoptotic, antiangiogenic, antimetstatic and mitogenic factor-inhibiting activities.

They are therefore particularly active as anticancer agents, in particular on breast cancers (hormone-dependent or non-hormone-dependent breast cancers) and on melanomas.

Said pharmaceutical composition is preferably administered systemically, such as subcutaneously, intravenously or orally.

The overall effective doses of the polymer(s) combined with at least one phenylcarboxylic acid salt of formula (I′), or of conjugate compounds of formula (III), can vary within large proportions, preferably ranging from 0.1 mg/kg per day to 200 mg/kg per day, at a rate of twice a week, and even more preferentially from 15 mg/kg per day to 150 mg/kg per day, at a rate of twice a week, and in particular when a conjugate compound of formula (III) is used.

The conjugate compounds of formula (III) as described above are novel compounds which, in its capacity, constitute another subject of the invention. Among the compounds of formula (III), the compounds of formula (IIIa) are preferred.

Among the compounds of formula (IIIa) preference is given to those in which a>0.5; b>0.15; c=0 or <0.8 and d<1.5.

Even more particularly, preference is given to the conjugate compounds of formula (IIIa) in which the ds values are as follows:

i) a=0.68; b=0.20; c=0 and d=0.14;

ii) a=0.64; b=0.43; c=0 and d=0.35;

iii) a=0.67; b=0.39; c=0 and d=0.35 (LS17-NaPAC);

iv) a=1; b=c=0 and d=0.1; and

v) a=1; b=c=0 and d=0.3.

The conjugate compounds of formula (III) as described above can be prepared according to conventional methods of esterification consisting, for example, in carrying out, in an organic solvent medium, an esterification reaction between at least one free hydroxyl function of the polymer and at least one phenylalkylcarboxylic acid derivative of formula (I) as described above, in the presence of an aliphatic or aromatic tertiary amine or of a coupling agent or of an acyl chloride or of an anhydride, so as to form a covalent bond between said derivative of formula (I) and said polymer, and thus to produce the compound of formula (III) expected.

This method of preparation may comprise a preliminary step of solubilization of the polymer, for example either by heating said polymer in the reaction solvent in the presence of lithium salts, or, only in the case of functionalized dextrans, by ion exchange on a column and neutralization with an aliphatic or aromatic tertiary amine, preferably that used as catalyst in the esterification reaction.

There are five factors which can influence the esterification:

1) the nature of the organic solvent: they are preferably chosen from dimethyl sulfoxide (DMSO), formamide, dimethylacetamide, dimethylformamide (DMF) and mixtures thereof. DMF is particularly preferred;

2) the nature of the tertiary amine or the coupling agent: the tertiary amines which can be used according to this method are preferably chosen from pyridine and alkyl derivatives thereof, triethylamine, tributylamine and N,N-dimethylaniline. Pyridine and triethylamine are particularly preferred. The amount of tertiary amine to be used can be calculated as a function of the amount of free hydroxyl functions in the polymer used. As coupling agent, use may, for example, be made of a carbodiimide, such as 1,1′-carbonyldiimidazole (CDI) or N,N′-dicyclohexyl-carbodiimide (DCC), at a temperature of between 0 and 60° C., the molar ratio of the phenylalkylcarboxylic acid derivative of formula (I) to the CDI being between 0.001 and 10, and the molar ratio of the CDI to the polymer possibly varying between 0.01 and 10. CDI is soluble in organic solvents such as DMSO, DMF, formamide, etc.

3) the reaction time;

4) the reaction temperature;

5) the number (or concentration) of free hydroxyl functions in the polymer used.

By way of example, the amount of free hydroxyl functions in the compounds of formula (II), in the form of a triethylamine salt, can be calculated according to the following method:

It is considered that each glucoside unit consists of three imaginary substitutable subunits. This gives:

R—OH molar mass: 54 g/mol

ROCH₂CONHCH_{2φ}(φ=phenyl) molar mass: 201 g/mol

ROCH₂COOHNET₃(Et=ethyl) molar mass: 213 g/mol

As a function of the mass of the dry product, the number of free hydroxyl functions is determined using the following equations:

(1)h×0.054+b×0.201+t×0.213=1 g

with [h/(h+b+t)]×3=ds_{OH}

[b/(h+b+t)]×3=ds_{b}

[t/(h+b+t)]×3=dsₐ

h: number of moles of free hydroxyls on the dextran derivative;

b: number of moles of N-benzylmethylene-carboxamide groups (ROCH₂CONHCH_{2φ}) on the compound of formula (II);

t: number of moles of ROCH₂COOHNEt₃ groups on the compound of formula (II);

ds_{b}: degree of substitution of N-benzylmethylenecarboxamide groups;

dsₐ: degree of substitution of methylcarboxylic groups;

dS_{OH}: amount of free OH.

Or(2): h+b+t=(3×h)/ds

which gives:

t=dsₐ/ds_{OH}

b=ds_{b}/ds_{OH}

These equations are then replaced in equation (1):

h[0.054+(dsₐ/ds_{OH})×0.213+(ds_{b}/ds_{OH})×0.201]=1

consequently,

h=1/[0.054+(dsₐ/ds_{OH})×0.213+(ds_{b}/ds_{OH)}×0.201]

For example, for a compound of formula (II) having the following degrees of substitution: ds_{b}=0.33; dsₐ=0.67; ds_{OH}=3−(ds_{b}+dsₐ)=2

The amount of free hydroxyl residues is therefore h=6.06 mmol/g.

The volumes of triethylamine (VI) and of the phenylalkylcarboxylic acid derivative of formula (I) used in the conjugation step (V2) are determined according to the following ratios:

V1:(N_{φCH2COCl}/N_{OH}) V2:(N_{φCH2COCl}/N_{NET3})

with

N_{φCH2COCl}: number of mmoles of phenylalkylcarboxylic acid derivative of formula (I)

N_{OH}: number of mmoles of hydroxyl group

N_{NET3}: number of mmoles of triethylamine.

In the conjugate compounds of formula (III), the degrees of substitution d of compounds of formula (I) can be determined by acidimetric assay, infrared spectrophotometry or UV-absorption spectrophotometry.

When the synthesis is finished, the conjugate compounds of formula (III) may optionally be purified according to conventional methods such as, for example, precipitation in alcoholic medium and/or ultrafiltration.

Besides the above arrangements, the invention also comprises other arrangements which will emerge from the following description, which refers to examples of synthesis of conjugate compounds of formula (III) according to the invention and to several examples of demonstration of the synergy of action between a dextran of formula (II) combined with or conjugated to a phenylalkylcarboxylic acid salt of formula (I), and also to the attached figures, in which:

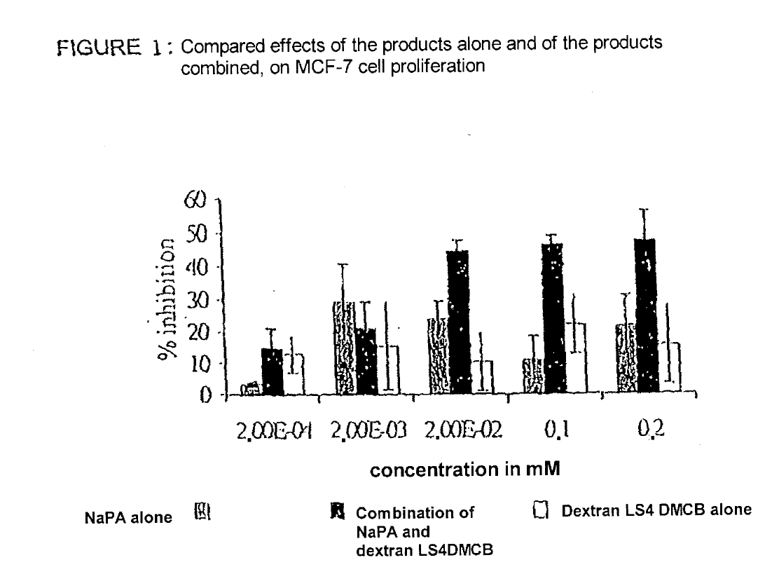

FIG. 1 represents the compared inhibitory effects of the products alone and of the products combined, on MCF-7 cell proliferation;

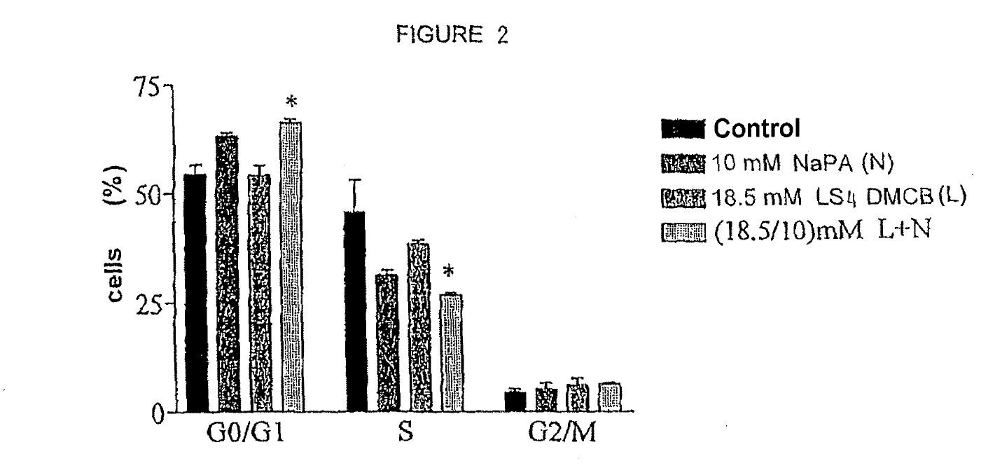

FIG. 2 shows the effects of NaPa in combination with LS4 DMCB, on the distribution of MCF-7ras cells within the three phases of the cell cycle (G₀/G₁/S and G₂/M);

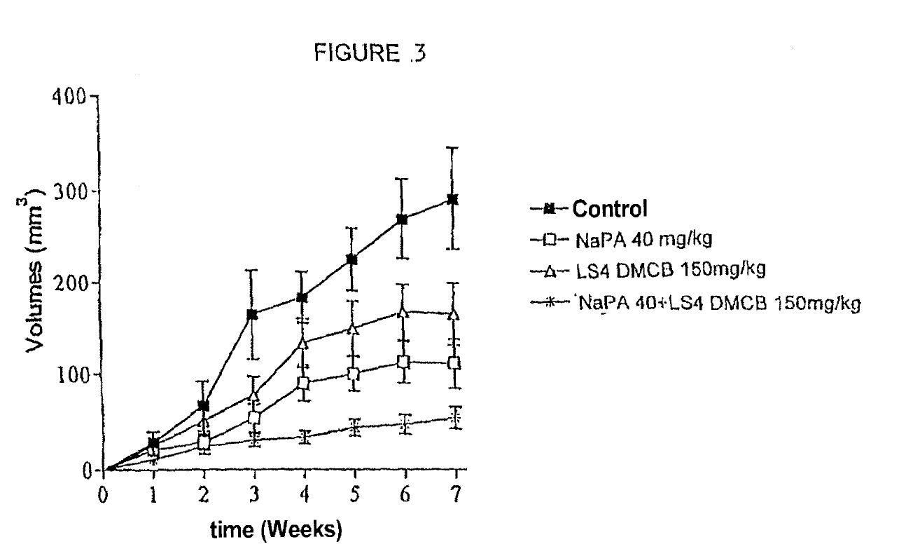

FIG. 3 represents the compared effects of the products alone and of the products combined, on the decrease in volume of tumors induced by inoculation of MCF-7ras cells in athymic mice; and

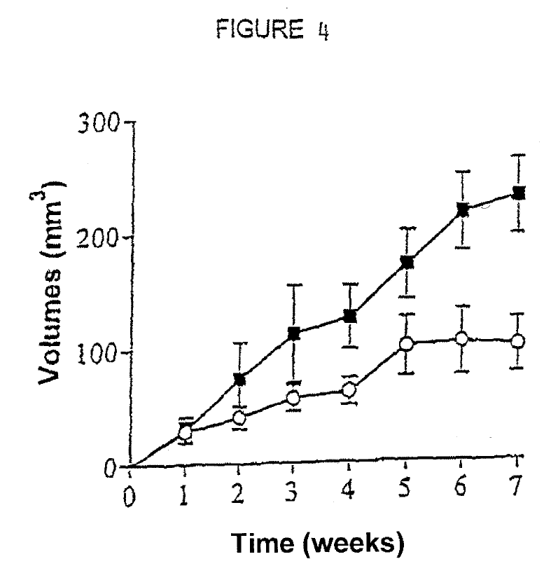

FIG. 4 represents the effects of a conjugate compound of formula (III) (NaPAC) on the decrease in volume of tumors induced by inoculation of MCF-7ras cells in athymic mice; and

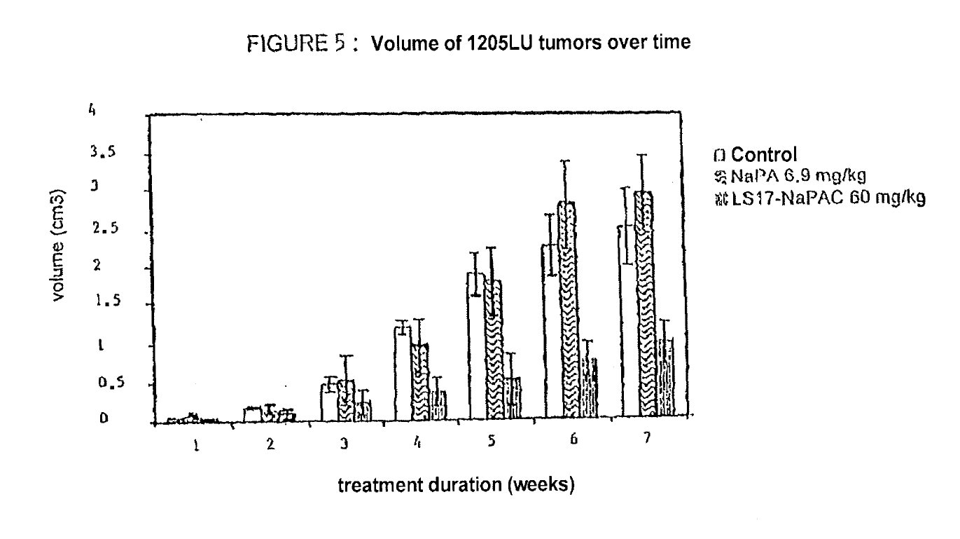

FIG. 5 shows the effects of NaPA alone and of LS17-NaPAC on the decrease in volume of tumors induced by inoculation of 1205LU cells in athymic mice.

It should be clearly understood, however, that these examples are given only by way of illustration of the subject of the invention of which they in no way constitute a limitation.

### EXAMPLE 1

### Synthesis of LS17-NaPAC

The synthesis of LS17-NaPAC first of all needed the precursor LS17 to be transferred into the form of a pyridine salt in order to be able to solubilize it in an anhydrous polar solvent, generally a Lewis base, such as DMF.

Transfer into the form of a Pyridine Salt

41 g of LS17 were dissolved in 774 ml of double-distilled water. The solution thus obtained was poured through a column containing 69.2 g of Purolite C100H ion exchange resin. The pH of the solution recovered at the resin column outlet was adjusted to 5.5 with pyridine. This solution was then frozen and lyophilized. Approximately 38 g of LS17 were obtained in the form of a pyridine salt (LS17-pyr) after drying in an oven under vacuum at 50° C. for 24 h.

Attachment of Phenylacetate

37 g of LS17-pyr obtained above in the preceding step were dissolved in 2.5 L of anhydrous DMF. The solution obtained was poured into a jacketed 5 L reactor under nitrogen. 42.8 ml of pyridine were then added thereto. In addition, a solution of phenylacetic acid chloride was prepared by mixing 35.2 ml of phenylacetic acid chloride in 352 ml of anhydrous DMF, and rapidly added to the mixture of the jacketed reactor with stirring. The reaction mixture was stirred for 1 hour.

The reaction was stopped by adding a 1M sodium hydroxide solution. The LS17-NaPAC obtained was then purified by tangential ultrafiltration using a Millipore system equipped with a membrane having a cutoff threshold of 5 000 g/mol. The purified solution was then concentrated, frozen and then lyophilized.

The LS17-NaPAC had the following degrees of substitution:

a=0.67; b=0.39; c=0 and d=0.35.

### EXAMPLES 2 TO 15

### Synthesis of Conjugate Compounds of Formula (III) in DMF

Dextran derivative used: LS4 DMCB in which a=0.67 and b=0.33.

Prior Step of Solubilization of LS4 DMCB

The dextran LS4 DMCB was solubilized, in the form of its tertiary amine salt, by cation exchange chromatography (IR-120 H⁺ resin). 4 amines were used: triethylamine, tributylamine, pyridine and N,N-di-methylaniline.

6 g of LS4 DMCB dextran derivative were dissolved in 200 ml of double-distilled water and then chromatographed on a column of IR-120 H⁺ resin (volume: 1.9 meq/ml; capacity: 10%). The product was chromatographed with a volume of double-distilled water representing two to three times the column volume. The product recovered was neutralized with a tertiary amine solution. The latter was prepared differently depending on the tertiary amine used. Since the tributylamine and the N,N-dimethylamine are immiscible in water, the neutralizing solution was prepared in ethanol. The final solution, with a volume of 1000 ml, was concentrated by rotary evaporation and then frozen and lyophilized.

The LS4 DMCB dextran thus solubilized was used to prepare the conjugate compounds of formula (III) hereinafter called NaPAC MF.

The conditions for synthesis are summarized in table I below. Before any synthesis, the dextran derivative is placed in an oven at 50° C. The dried dextran is dissolved in DMF with stirring in a jacketed reactor maintained at the reaction temperature. A solution of the tertiary amine at 10% (v:v) in DMF is added using a syringe. The phenylacetic acid chloride, prepared in the same way, is then added using a syringe. After one hour, the reaction is stopped by adding 150 ml of double-distilled water. The pH is adjusted to 7 with a 0.1M sodium hydroxide solution.

The solution obtained is purified in three steps:

evaporation to dryness in a rotary evaporator in order to remove the solvents;

extraction with ether in basic medium: the solid obtained after evaporation is redissolved in 50 ml of double-distilled water. The pH solution thus obtained is adjusted to 10 with a dilute sodium hydroxide solution, and the solution is then washed with diethyl ether (3×20 ml) in a separating funnel. The aqueous phase is neutralized with dilute hydrochloric acid and then ultrafiltered;

ultrafiltration: the volume of the aqueous phase is adjusted to 2 liters with double-distilled water, and then ultrafiltered with a 2M NaCl solution (20 L) and then with double-distilled water. The retentate is frozen and then lyophilized.

The products of examples 2 to 9, 11 and 15 were purified and, after purification, the products obtained had the following characteristics:

| | Conjugate | | Degree of |
|---|---|---|---|
| | compound of | | substitution of |
| Example | formula (III) | Yield | NaPA (d) |
| 2 | NaPAC MF3 | 77% | 0.24 ± 0.05 |
| 3 | NaPAC MF4 | 82% | 0.68 ± 0.1 |
| 4 | NaPAC MF5 | 70% | 0.07 ± 0.04 |
| 5 | NaPAC MF6 | 91% | 0.71 ± 0.1 |
| 6 | NaPAC MF7 | 71% | 0.43 ± 0.05 |
| 7 | NaPAC MF8 | 84% | 0.53 ± 0.08 |
| 8 | NaPAC MF9 | 81% | not calculatable |
| 9 | NaPAC MF10 | 95% | 0.22 ± 0.09 |
| 11 | NaPAC MF12 | 90% | 0.27 ± 0.01 |
| 15 | NaPAC MF14 | 79% | 0.28 ± 0.06 |

TABLE I
CONDITIONS FOR SYNTHESIS IN DMF

| | | | | | | | | | | Vol- | | | |
|---|---|---|---|---|---|---|---|---|---|---|---|---|---|
| | | | | Concen- | Amount | | Amount | | | ume* | Volume* | | |
| | Compound | | Tem- | tration | of free | Amount | of Net3 | | | of | of | Volume | |
| | of formula | Tertiary | pera- | of free | OH in | of RCOCl | in mol | | | RCOCl | amine | of DMF | Final |
| Ex. | (III) | amine | ture | OH | mol | in mol | V1 | V1 | V2 | in ml | in ml | in ml | volume |
| 2 | NaPAC MF3 | pyridine | 30° C. | 0.12 M | 5.87.10⁻³ | 5.87.10⁻³ | 1.17.10⁻² | 1 | 2 | 7.8 | 9.8 | 31.3 | 48.9 |
| 3 | NaPAC MF4 | pyridine | 30° C. | 0.06 M | 5.93.10⁻³ | 5.93.10⁻³ | 1.19.10⁻² | 1 | 2 | 7.9 | 9.8 | 81.1 | 98.8 |
| 4 | NaPAC MF5 | triethyl- | 30° C. | 0.12 M | 6.90.10⁻³ | 6.90.10⁻³ | 1.38.10⁻² | 1 | 2 | 9.2 | 18.9 | 29.4 | 57.5 |
| | | amine | | | | | | | | | | | |
| 5 | NaPAC MF6 | triethyl- | 30° C. | 0.06 M | 6.24.10⁻³ | 6.24.10⁻³ | 1.25.10⁻² | 1 | 2 | 8.2 | 17.0 | 104 | 129.2 |
| | | amine | | | | | | | | | | | |
| 6 | NaPAC MF7 | triethyl- | 30° C. | 0.12 M | 4.93.10⁻³ | 4.93.10⁻³ | 9.86.10⁻³ | 1 | 2 | 6.5 | 23.5 | 11.0 | 41.0 |
| | | amine | | | | | | | | | | | |
| 7 | NaPAC MF8 | triethyl- | 30° C. | 0.06 M | 4.83.10⁻³ | 4.83.10⁻³ | 9.66.10⁻³ | 1 | 2 | 6.4 | 23.0 | 51.0 | 80.4 |
| | | amine | | | | | | | | | | | |
| 8 | NaPAC MF9 | N,N- | 30° C. | 0.12 M | 3.73.10⁻³ | 3.73.10⁻³ | 7.46.10⁻³ | 1 | 2 | 5.0 | 9.5 | 16.6 | 31.1 |
| | | diethyl- | | | | | | | | | | | |
| | | amine | | | | | | | | | | | |
| 9 | NaPAC Mf10 | N,N- | 30° C. | 0.06 M | 4.42.10⁻³ | 4.42.10⁻³ | 8.84.10⁻³ | 1 | 2 | 5.8 | 16.8 | 51.0 | 73.6 |
| | | diethyl- | | | | | | | | | | | |
| | | amine | | | | | | | | | | | |
| 10 | NaPAC MF11 | pyridine | 50° C. | 0.12 M | 5.93.10⁻³ | 5.93.10⁻³ | 1.19.10⁻² | 1 | 2 | 8.0 | 9.8 | 51.3 | 69.1 |
| 11 | NaPAC MF12 | pyridine | 50° C. | 0.06 M | 5.83.10⁻³ | 5.83.10⁻³ | 1.17.10⁻² | 1 | 2 | 7.7 | 9.6 | 79.8 | 97.1 |
| 12 | NaPAC MF13 | triethyl- | 50° C. | 0.12 M | 6.11.10⁻³ | 6.11.10⁻³ | 1.22.10⁻² | 1 | 2 | 8.1 | 16.7 | 26.1 | 50.9 |
| | | amine | | | | | | | | | | | |
| 13 | NaPAC MF14 | triethyl- | 50° C. | 0.06 M | 6.65.10⁻³ | 6.65.10⁻³ | 1.33.10⁻² | 1 | 2 | 8.8 | 18.1 | 84.0 | 110.9 |
| | | amine | | | | | | | | | | | |
| 14 | NaPAC MF15 | tributyl- | 50° C. | 0.12 M | 4.93.10⁻³ | 4.93.10⁻³ | 9.86.10⁻² | 2 | 6.5 | 23.5 | 11.0 | 41.0 | |
| | | amine | | | | | | | | | | | |
| 15 | NaPAC MF16 | tributyl- | 50° C. | 0.06 M | 4.88.10⁻³ | 4.88.10⁻³ | 9.76.10⁻² | 1 | 2 | 6.4 | 23.3 | 51.6 | 81.3 |
| | | amine | | | | | | | | | | | |

*:Solution at 10% (in DMF)
V1: (ROCOCl]/[OH)
V2: (RCOCl]/[amine)
MM (RCOCl): 154.6 g/mol
MM (NEt3) 101.18 g/mol
MM (pyridine): 79.1 g/mol
MM (N,N-diethylamine): 121.8 g/md
d (RCOCl) 1.1595 g/ml
d (NEt3) 0.724 g/ml
d (pyridine) 0.978 g/ml
d (N,N-diethylamine) 0.956 g:ml

### EXAMPLE 16

### Demonstration of the Synergy of Action In Vitro Between a Dextran of Formula (II) Combined with or Conjugated to a Phenylalkylcarboxylic Acid Salt of Formula (I′) on the Inhibition of MCF-7 Cell Proliferation

I) Materials and Methods

1. The MCF-7 Tumor Cell Line

The MCF-7 tumor line is a line of estrogen-sensitive epithelial cells originating from pleural metastases of an infiltrating ductule breast adenocarcinoma. They are cultured in Dulbecco's Modified Eagle Medium (DMEM), supplemented with 10% of fetal calf serum (FCS), 1% of glutamine and 1% of sodium pyruvate. The cells are stored in cryotubes in frozen form in liquid nitrogen. The cryotubes are partially thawed in a water bath at 37° C. 2 ml of complete culture medium are added and the cell suspension is then mixed. Culture flasks (COSTAR® T25) are prepared with culture medium beforehand. The cell suspension is added to the flasks and placed in an incubator at 37° C. with 5% of CO₂.

When the cells are virtually confluent, the monolayer is washed with 2 ml of phosphate buffer (PBS), once, without calcium and magnesium. Once the PBS has been removed, 2 ml of trypsin are deposited onto the monolayer in order to detach the cells. Each trypsinization corresponds to a numbered passage. 8 ml of complete culture medium are subsequently added in order to stop the action of the trypsin.

After homogenization, the cell suspension is centrifuged for 3 to 4 minutes at 1 000 rpm. The pellet containing the cells is recovered and then diluted in 10 ml of complete culture medium. The number of cells per ml is determined using a Malassez cell, in order to seed a precise volume of cells in the 75 cm² culture flasks (T75, COSTAR®). The cells are cultured at 37° C. in a humid atmosphere containing 5% of CO₂.

2. Proliferation Kinetics

The cells are seeded randomly in a portion of 15 000 cells per ml and per well, in 24-well plates (COSTAR®). Daily samples are taken in a proportion of 4 wells per day. The number of cells contained in each of the wells is determined by Coulter Counter ZM® counting so as to have three measurements per well. Based on these data, a curve for MCF-7 cell proliferation is plotted as a function of time.

3. Cell Growth Assay

Cells sampled in the exponential growth phase are seeded in 24-well plates in a proportion of 15000 cells per ml and per well, this being in a DMEM medium supplemented with 10% FCS, 1% of sodium pyruvate and 1% of glutamine.

After incubation for 24 hours, the various concentrations of products to be tested are added in the proportion of 4 wells per concentration. These products are diluted in a DMEM medium containing 2% of FCS, 1% of sodium pyruvate and 1% of glutamine, so as to obtain the desired concentrations. The negative control is prepared without the addition of product.

After incubation for 72 hours, the cells are detached and then counted using a Coulter Counter ZM® set on the parameters predefined for MCF-7 cells. The diameter (threshold) of detection is 5 for an attenuation of 32. The latter determines the number of particles of desired diameter contained in the sample. By taking into account the dilution made, the number of cells per ml of initial cell suspension is determined from this value. The percentage inhibition I can then be determined from these results using the following equation:

%I=[1−(net increase in treated cells/net increase in untreated cells)]×100

The net increase in untreated cells corresponds to the number of cells contained in the control wells (DMEM medium containing 2% FCS) subtracted with the number of cells at D₀ (number of cells at the time the products are added to the wells).

The net increase in treated cells corresponds to the number of treated cells at D₇₂ minus the number of cells at D₀.

4. MTT Growth Assay

MTT (3-(4,5-dimethylthiazol-2-yl)-2,5-diphenyl-tetrazolium bromide) has the property that it can be reduced in alkaline medium, thus giving a colored compound, blue formazan. Mitochondria, by virtue of their membrane-bound dehydrogenases, are capable of carrying out this reduction. As a result, the number of functional mitochondria, and indirectly the number of live cells, can be quantified as a function of the intensity of the coloration.

Cells sampled in the exponential growth phase are seeded in 96-well plates in a proportion of 5 000 cells per 200 μl, this being in a DMEM medium supplemented with 2% of FCS, 1% of glutamine and 1% of sodium pyruvate. After incubation for 24 hours, the various concentrations of products to be tested are added in a proportion of 3 wells per concentration. A negative control is prepared without the addition of product.

After incubation for 72 hours, the cells are washed twice with a buffered saline solution and then 100 μl of MTT are added to each well. After 4 hours of incubation, 100 μl of DMSO are added to the wells before reading the plate 570 nm using a plate reader.

The percentage inhibition I is determined using the equation previously described. The net increase in untreated cells this time corresponds to the cell density contained in the control wells minus that of D₀. As regards the net increase in treated cells, it corresponds to the treated cell density at D₇₂ subtracted with that of D₀.

II) Results

1. Kinetics of MCF-7 Cell Proliferation

These kinetics make it possible to characterize the growth of MCF-7 cells. This growth, plotted as semi-logarithmic coordinates, breaks down into three phases:

The lag phase: this corresponds to the time required for the cells to adapt to this new medium and thus to allow them to adhere to the surface. This phase is characterized by the lag time (T_{L}).

The exponential phase: this corresponds to the time required for a cell to divide by mitosis and thus to give two daughter cells. This phase is characterized by a doubling time (T_{D}) determined by virtue of the following equation:

T_{D}=[(t−t₀)×log 2]/(Log N−Log N₀)

The stationary growth phase: this represents the cells at confluence. These cells occupy the entire surface available to them and can no longer grow. A depletion of the medium and an increase in metabolite concentration result in apoptosis.

The doubling time and also the lag time are not identical for all cells. As regards MCF-7 cells, the doubling time is very rapid, of the order of 18 hours, and as for the lag time, it is of the order of 24 hours.

2. Effects of the Products to be Tested on the MCF-7 Line

The aim of these assays is to demonstrate that combining or conjugating at least one dextran and at least one phenylcarboxylic acid salt of formula (I′) results in a synergy of action relative to the activity of each of the products used alone. For this, the products alone (sodium phenylactate: NaPA and LS4 DMCB dextran derivative), and also the corresponding combined and conjugated products were tested on the MCF-7 tumor cell line. The combination consists of a mixture of the dextran LS4 DMCB with NaPA in the same solution. As for the conjugation, this represents the attachment on NaPA to the dextran LS4 DMCB via a covalent bond. The conjugate compounds of the examples of syntheses 3, 6 and 8 (NaPAC MF3: d=0.24, NaPAC MF6: d=0.71, and NaPAC MF8: d=0.53) were thus tested.

a) Effect of NaPA Alone

The effect of NaPA alone on MCF-7 cells was first of all observed by cell counting, on a range of concentrations of from 1 mM to 50 mM. NaPA induces an antiproliferative effect which increases as a function of the concentration. When these concentrations are greater than 30 mM, the inhibition of cell proliferation exceeds 100%, thus reflecting a cytotoxic effect of the product. The IC₅₀ (minimum dose causing arrest of proliferation of 50% of cells) is obtained for a concentration of 5 mM.

The effect of NaPA at concentrations less than 1 mM was evaluated. For practical reasons, the percentage inhibition was quantified by the calorimetric method (MTT). At these concentrations, NaPA does not appear to have a significant effect on MCF-7 cells.

b) Effect of the Dextran LS4 DMCB Alone

The proliferation-inhibiting effect, for concentrations ranging from 1 μg/ml to 1500 μg/ml, was evaluated by direct counting of the cells on a Coulter Counter ZM®. This product does not appear to have an inhibitory effect on MCF-7ras cells, for concentrations not exceeding 1 000 μg/ml.

For higher concentrations, the experiments carried out by the MTT calorimetric method show that the percentage inhibition reaches a plateau with a maximum of around 30% inhibition.

c) Compared Effects of the Products Alone and of the Products Combined

The compared experiments are carried out with the MTT calorimetric method.

The combination of the dextran LS4 DMCB at 1000 μg/ml with concentrations of NaPA ranging from 0.2 μM to 0.2 mM (which corresponds to concentrations of NaPA, when they are expressed by weight, of 0.33 μg to 0.33 mg) makes it possible to obtain an inhibition of between 40 and 50% of the proliferation of MCF-7 tumor cells (FIG. 1). This same inhibition is obtained for a concentration of free NaPA of 5 mM. The combination of at least one dextran and at least one phenylalkylcarboxylic acid salt of formula (I) in accordance with the invention therefore exhibits a synergy of action on the inhibition of MCF-7 cell proliferation, making it possible to reduce by at least 6-fold the amount of NaPA used.

d) Compared Effects of the Products Alone and of the Products Conjugated

The compared experiments are carried out by cell counting on a Coulter Counter ZM®.

Just as for comparison c), the effect of the conjugated products on the inhibition of MCF-7 cell proliferation is much greater than the effects of products used alone.

NaPAC MF6, with a degree of substitution of 0.8, contains 2.27 mM of NaPA per mg of product. The maximum effect of NaPAC MF6 is observed for a concentration of 250 μg/ml. This represents an equivalent concentration of 0.56 mM of free NaPA.

With NaPA alone, this inhibition is obtained for a concentration of 50 mM.

Identical results were obtained with NaPAC MF3 and NaPAC MF8.

Consequently, conjugating at least one dextran of formula (II) and at least one phenylalkylcarboxylic acid derivative of formula (I) in accordance with the invention gives a synergy of action on the inhibition of MCF-7 cell proliferation which makes it possible to decrease by at least 10-fold the concentration of NaPA required to significantly inhibit the growth of MCF-7 cells.

### EXAMPLE 17

### Demonstration of the Synergy of Action Between a Dextran of Formula (II) and a Phenylcarboxylic Acid Salt of Formula (I′) on the Inhibition of MCF-7ras Cell Proliferation In Vitro

I) Materials and Methods

1 Non-Hormone-Dependent Human Breast Cancer Cell Model

MCF-7 cells transfected with the Ha-ras oncogene (MCF-7ras cells) were cultured in DMEM medium supplemented with 10% of FCS, 2 mM of L-glutamine, 1 mM of sodium pyruvate, 50 units/ml of penicillin and 50 μg/ml of streptomycin, at a temperature of 37° C. and in a humid atmosphere with 5% of CO₂.

2. MTT Growth Assay

This was carried out in 96-well microplates under the same conditions as those given above in example 17.

3. DNA Analysis

In order to study the DNA synthesis, the MCF-7ras cells were labeled by incubation with 1 μCi of [H³]-thymidine (specific activity 82 Ci/mmol; Amersham Life Science Inc., UK) or with 10 uM of bromodeoxy-uridine (BrdU, Boehringer France) for 6 hours in a humid atmosphere containing 5% of CO₂. The cells incubated with [H³]-thymidine were harvested using a Skatron semi-automatic harvesting system (Skatron, Lier, Norway). After filtration through a glass fiber filter, the incorporated radioactivity was measured. After fixing cells and denaturing the BrdU-labeled DNA, 100 μl/well of anti-BrdU-POD serum (Boehringer, France) were added. The plates were incubated for 30 min. The BrdU/anti-BrdU-POD complex was detected by a reaction of the ELISA type, the reaction products being quantified by measuring the absorbance at 490 nm using a microplate reader (Biorad, France). The results of the ELISA assay correlate with the values obtained with the [H³]-thymidine incorporation assay.

4. Conditioned Experimental Medium

This medium is used to study the action of the tested products on the inhibition of the action of cellular growth factors.

MCF-7ras cells (5×10⁶) were cultured up to 80% confluency in a DMEM medium supplemented with 10% of FCS, washed twice with PBS buffer and then incubated in DMEM medium for 24 hours at 37° C. The medium thus conditioned by the MCF-7ras cells (conditioned medium) was then harvested, centrifuged and then stored at −80° C. before use.

BALB/c3T3 fibroblast cells (10⁵ cells) were placed in culture in DMEM supplemented with 10% of FCS in 24-well plates (Polylabo, France) and cultured until confluency, washed twice with DMEM and incubated for 24 hours in serum-free DMEM medium containing 0.1% of BSA (Sigma, St. Louis, Mo.).

After this treatment, the cells were quiescent. The medium was then replaced with a mixture of 0.75 ml/well of medium conditioned by MCF-7ras cells and 0.25 ml/well of DMEM in the presence or absence of the products to be tested (NaPA and LS4 DMCB) at various concentrations. After treatment for 48 hours, the cells were counted using a cell counter (Coulter).

5. Cell Cycle Analysis

To analyze the cell cycle, the cells were seeded in plates in a proportion of ×10⁴ cells/ml and per well in DMEM medium supplemented with 2% of FCS. After culturing for 24 hours, the various tested products were added.

After 72 hours, the MCF-7ras cells were incubated in the presence of BrdU for 4 hours. The cells were then washed with PBS buffer and fixed with 70% ethanol. The BrdU incorporated into the cells is revealed with anti-BrdU conjugated to fluorescein isothiocyanate (Boehringer Mannheim). The cells were then centrifuged and resuspended in a staining solution containing 50 μl/ml of propidium iodide (Boehringer Mannheim) for 10 min. The stained cells are then analyzed with a FACScan (Coulter).

6. Evaluation of Apoptosis

Several times over time, the cell viability was determined by trypan blue exclusion. The same cells were centrifuged and washed with an annexin buffer (Boehringer Mannheim). Translocation of phosphatidylserine was determined with FITC-labeled annexine V and, after treatment with propidium iodide, the cells were analyzed using a FACScan (Coulter).

7. Products Tested

In this example, the effects of the following products, alone or in combination, at various concentrations, were studied on MCF-7ras cell proliferation after incubation for 72 hours:

NaPA sold by the company Seratec was dissolved in distilled water;

dextran derivatives LS4 DMCB, DMCB7 and LS17.

The structure of DMCB7 is described in the article by R. Baghéri-Yarmand et al., 1994, In Vitro Cell. Dev. Biol. 30A, 8822-8824.

II) Results

The results obtained show that, after treatment for 72 hours, NaPA used alone inhibits MCF-7ras cell proliferation as a function of the dose used (70% inhibition at 30 and 40 mM). The dextran LS4 DMCB inhibits MCF-7ras cell proliferation with little difference as a function of the dose used, maximum inhibition (35%) occurs at a dose of 18.5 mM. The dextran DMCB7 inhibits MCF-7ras cell proliferation as a function of the dose used, with 60% inhibition at a concentration of 7 mM. It is noted that the dextran LS4 DMCB is more effective than DMCB7 on tumor cell proliferation at doses of less than 4 mM.

The combination of the dextran LS4 DMCB or the dextran DMCB7 and NaPA was analyzed using the dose-effect curves for each product used alone (see tables II, III and IV below). The dextran DMCB7, at increasing doses, was tested alone or in combination with a constant dose of NaPA of 5 mM (see table II below). The dextran LS4 DMCB, at increasing doses, was tested alone or in combination with increasing doses of NaPA (see table III below).

The same experiment was carried out with the dextran LS17, and the results are given in table IV below.

The percentage inhibition I was calculated as described above in example 17.

The effect of synergy between the dextrans DMCB7 or LS4 DMCB or LS17 and NaPA was calculated using the following isobolic equation (T. Mosmann, J. Immunol. Methods, 1983, 65, 55-63):

D=A_{c}/Aₑ+B_{c}/Bₑ

in which A_{c} and B_{c} correspond, respectively, to the concentration of dextran derivative and NaPA used in combination, and Aₑ and Bₑ correspond to the concentration of each of these two compounds used alone and inducing the same effect; D is the combination index and Dₘ is the mean combination index. When D or Dₘ is less than 1, then the combination of the two products has a synergistic effect. When D or Dₘ=1 or is greater than 1, then the combination of the two products has an additive effect or an antagonistic effect respectively.

When the concentration of dextran derivative used alone (Aₑ), making it possible to obtain a percentage inhibition close to that which is obtained when the dextran derivative is used in combination with NaPA, is very high and such that it cannot be determined precisely, then the ratio A_{c}/Aₑ tends toward zero and, consequently, D=B_{c}/Bₑ. Such concentrations of dextran derivatives (Aₑ) have been indicated in tables II, III and IV below as simply being greater than the concentration given for A_{c}.

Each experiment was repeated 3 times so as to be able to calculate the statistical significance (P) of the combination index (D or Dₘ) compared to the additive combination index for the effects (D=1) using the Student's t test.

TABLE II

| DMCB7 | NaPA | | DMCB7 | NaPA | | Dₘ: mean |
|---|---|---|---|---|---|---|
| A_{c} in | B_{c} in | % | A_{c} in | B_{c} in | D: combination | combination |
| mM | mM | inhibition I | mM | mM | index | index |
| 0.05 | 5 | 10 | 0.4 | 1 | 5.12 | >2 |
| 0.3 | 5 | 20 | 2.5 | 5 | 1.12 | 1.5 |
| 1 | 5 | 38 | 3.85 | 10.7 | 0.71^{a} | 0.54^{a} |
| 1.27 | 5 | 42 | 4.5 | 12 | 0.66^{a} | 0.56^{a} |
| 1.7 | 5 | 45 | 5 | 13 | 0.72^{a} | 0.5^{a} |
| 2 | 5 | 50 | >7.5 | 20 | 0.25^{a} | 0.58^{a} |
| 5 | 5 | 60 | >7.5 | 29 | 0.17^{a} | 0.17^{a} |
| 7 | 5 | 70 | >7.5 | 36 | 0.14^{a} | 0.14^{a} |

^{a}P < 0.05

TABLE III

| LS4 | NaPA | % | LS4 | NaPA | D: | Dₘ: mean |
|---|---|---|---|---|---|---|
| A_{c} in | B_{c} in | inhibi- | A_{c} in | B_{c} in | combination | combination |
| mM | mM | tion I | mM | mM | index | index |
| 3.7 | 0.75 | 51.4 | >18.5 | 22 | 0.03^{a} | 0.07^{a} |
| 7.4 | 1.5 | 54.8 | >18.5 | 23 | 0.065^{a} | 0.11^{a} |
| 14.8 | 3 | 55 | >18.5 | 23.3 | 0.13^{a} | 0.22^{a} |
| 18.5 | 4 | 60.2 | >18.5 | 29.3 | 0.14^{a} | 0.26^{a} |

^{a}: P < 0.05

TABLE IV

| LS17 | NaPA | % | LS17 | NaPA | D: | Dₘ: mean |
|---|---|---|---|---|---|---|
| A_{c} in | B_{c} in | inhibi- | Aₑ in | Bₑ in | combination | combination |
| mM | mM | tion I | mM | mM | index | index |
| 1.53 | 0.5 | 0 | 0 | 0 | — | — |
| 3 | 1 | 16 | 7.5 | 0.76 | 0.54^{a} | 0.32^{a} |
| 6 | 2 | 17.3 | >12 | 0.83 | 2.4 | 1.6 |
| 12 | 4 | 17.3 | >12 | 0.83 | 4.8 | 8.48 |

^{a}: P < 0.05

The results given in table II show a synergy of action of the combination on the inhibition of cell proliferation (70% inhibition) for 5 mM of NaPA and concentrations of DMCB7 of greater than 1. Concentrations of DMCB7 of less than 1 mM induce an additive effect.

The results given in table III show a synergy of action of NaPA (0.75 mM to 4 mM) combined with LS4 DMCB (from 3.7 to 18.5 mM) on the inhibition of cell proliferation. This synergy of action makes it possible to obtain an IC₅₀ (minimum concentration inducing 50% inhibition) of 4 mM, whereas this IC₅₀ is five times higher when NaPA is used alone.

The results given in table IV show that a synergy of action between LS17 and NaPA is demonstrated for the respective concentrations of 3 and 1 mM.

Consequently, the combination of at least one phenylalkylcarboxylic acid salt of formula (I′) and at least one dextran exhibits an effect of synergy on the inhibition of tumor cell proliferation.

Analysis of the distribution of MCF-7ras cells treated with NaPA alone (10 mM), the dextran LS4 DMCB alone (18.5 mM) and a combination thereof (NaPA: 10 mM; LS4 DMCB: 18.5 mM) in the various phases of the cell cycle was carried out after 48 hours of treatment.

The results are given in FIG. 2.

These results show that treatment with NaPA induces blocking of the cells in the G₀/G₁ phase (G₀: resting phase; G₁: interphase) concomitant with blocking of DNA replication. When the dextran LS4 DMCB and NaPA are combined, the inhibition of DNA replication and the distribution of the cells in the G₀/G₁ phase are increased. The combination of at least one dextran and at least one phenylcarboxylic acid salt of formula (I′) therefore makes it possible to block the MCF-7ras cells in the G₀/G₁ phase and thus reinforces the cytostatic effect of each of the products used alone.

With regard to apoptosis, it has been shown (Adam et al., Cancer Res., 1997, 57, 1023-1029) that NaPA induces apoptosis alone or in combination with tamoxifen.

In the context of the present invention, it was observed that the combination of NaPA at 5 mM with DMCB7 or LS4 DMCB induces greater apoptosis of the MCF-7ras cells than the products used separately.

### EXAMPLE 18

### Study of the Antitumor Effect In Vivo of a Phenylalkylcarboxylic Acid Salt of Formula (I′) Combined with a Dextran of Formula (II) on a Non-Hormone-Depedent Breast Tumor (MCF7ras Cells)

NaPA sold by the company Seratec was dissolved in distilled water.

In this example, the dextran derivative LS4 DMCB, as described above, was used.

Fifty 3-week-old female athymic mice from the Laboratoire Harlan (Gannat, France) were kept in a temperature-controlled room and subjected to a cycle of light/dark alternating every 12 hours.

The mice were given water and food.

Cells of the MCF-7ras tumor line as described above in example 17 were cultured in a DMEM medium supplemented with 10% of FCS until 80% confluency was reached. After centrifugation, the cells were resuspended in DMEM supplemented with 10% of FCS.

Each mouse was then inoculated subcutaneously with these cells, in the proximity of the mammary gland, in a proportion of 5×10⁶ cells per animal.

After three weeks of inoculation, 70% of the mice infected exhibited a palpable subcutaneous tumor.

The volume of the tumors (V) was calculated in the following way:

V=(4/3)π(r₁)²)r₂); r₁ representing the smallest radius of the tumor and r₂ the largest radius of the tumor.

Four weeks after inoculation of the tumor cells, the mean volume of the tumors was approximately 10 mm³.

Four weeks after inoculation, solutions of NaPA (40 mg/kg) or of dextran LS4 DMCB (150 mg/kg) or of a combination of NaPA (40 mg/kg) and dextran LS4 DMCB (150 mg/kg), prepared in 0.1 ml of sodium chloride, were injected into the mice, twice a week, for 7 weeks for the solutions of NaPA alone and of dextran LS4 DMCB alone. The injections of the combination of NaPA and dextran LS4 DMCB were stopped after six weeks.

For each solution tested, 10 mice chosen randomly from the 50 mice initially inoculated were subjected to the treatment.

During the same period, a group of 10 mice were given an injection of 0.1 ml of sodium chloride to act as a control group.

The volume of each tumor was measured once a week.

In FIG. 3, the results are given in the form of mean with a 95% confidence interval.

The various statistical comparisons were carried out using ANOVA and Mann-Whitney tests in a multivariable linear model.

The results obtained appear in FIG. 3, in which the tumor volume in mm³ is expressed as a function of time in weeks, the closed squares correspond to the results obtained on the group of control mice, the open squares correspond to the results obtained on the group of mice to which NaPA alone was administered, the rhombs correspond to the results obtained on the group of mice to which the dextran LS4 DMCB alone was administered and the asterisks correspond to the results obtained on the group of mice to which the mixture NaPA and dextran LS4 was administered.

After 7 weeks of treatment, it is observed that injection of NaPA inhibited the tumor cell growth by 59% (P=0.049).

After 4 weeks of treatment, it is observed that injection of dextran LS4 DMCB inhibited the tumor cell growth by 38% (P=0.0084); however, the growth of the cells began again after 7 weeks (P>0.05).

Injection of the solution containing the combination of NaPA and dextran LS4 DMCB led to an 83% inhibition of the tumor cell growth, and no recommencement of cell proliferation was then observed (P=0.046).

No apparent toxicity of these products was observed for the duration of the treatment.

All of these results show that the combination of at least one phenylalkylcarboxylic acid salt of formula (I) and at least one dextran derivative of formula (II) exhibits a synergy of action which makes it possible to very considerably decrease the tumor cell growth, this decrease being clearly greater than the decrease in tumor cell growth observed with each of the products used separately. These in vivo results confirm the synergy of action on the inhibition of cell proliferation observed in vitro.

### EXAMPLE 19

### Study of the Antitumor Effect In Vivo of a Phenylalkylcarboxylic Acid Salt of Formula (I) Conjugated to a Dextran of Formula (II) on a Non-Hormone-Dependent Breast Tumor (MCF-7ras Cells)

In this example, the conjugate compound of formula (III) synthesized in example 1 above was used (LS17-NaPAC).

Two different doses of LS17-NaPAC were tested on female athymic mice, according to the protocol described above in example 18:

The mice were treated with doses of 15 mg/kg of LS17-NaPAC and no apparent toxicity was observed. The results obtained are given in FIG. 4, in which the closed squares represent the results obtained on the group of control mice having received no treatment and the circles represent the results obtained on the group of mice having received doses of 15 mg/kg of LS17-NaPAC.

These results show that the LS17-NaPAC at the dose of 15 mg/kg inhibits by 57% the growth of MCF-7ras tumors (P=0.0069) after 6 weeks of treatment, in the same way as the NaPa used alone at a dose of 40 mg/kg (see example 18 above), whereas the dextran LS17 used alone has no effect on MCF-7ras cell proliferation in vitro. These results show that the effective doses of the compounds used in the conjugate form are lower than those of the compounds used in the combined form and that, consequently, the synergy of action is even more marked in the case of the conjugate compounds of formula (III).

### EXAMPLE 20

### Comparative Study In Vivo of the Antitumor Effect of LS17-NaPAC and of NaPA Alone at Equivalent Concentrations on a Human Melanoma (1205LU Cells)

A group of 14 female athymic mice were subcutaneously inoculated, according to the protocol described above in example 17, with 1205LU human melanoma cells, in a proportion of 10⁶ cells per animal. After four days of inoculation, 100% of infected mice exhibited a palpable subcutaneous tumor.

For a period of five weeks, and at a rate of twice a week, a first group of 4 mice were given an injection of 100 μl of sodium chloride so as to serve as a control group, a second group of 4 mice were given an injection of LS17-NaPAC at a dose of 60 mg/kg/100 μl, and a third group of 6 mice were given an injection of NaPA at a dose of 6.9 mg/kg/100 μl.

It is important to note that a dose of 60 mg/kg/100 μl of LS17-NaPAC contains 6.9 mg/kg/100 μl of NaPA.

The volume of each tumor was measured before the first injection and then once a week for the five weeks of treatment. It was calculated according to the equation defined above in example 17.

The results were subjected to a nonparametric test and are given in FIG. 5. It can be noted that NaPA used alone does not appear to have a significant effect on tumor growth. On the other hand, LS17-NaPAC used at a dose of 60 mg/kg inhibits the growth of the tumors (p=0.001) when it is compared to NaPA.

Consequently, the conjugation of NaPA with LS17-DMCB is more effective than NaPA used alone, and at an equivalent concentration, on the decrease in volume of tumors obtained from 1205LU melanoma cells.

Furthermore, at the end of treatment, the mice were sacrificed so as to be able to determine the mass of each of the tumors. The results obtained show that the curves expressing the mass of the tumors taken from the untreated mice (control group) and that of the tumors taken from the mice treated with NaPA alone at a dose of 6.9 mg/kg or with LS17-NaPAC at a dose of 60 mg/kg confirm the results obtained previously. There is no significant difference between the mass of the tumors taken from the untreated mice and that of the tumors taken from the mice treated with NaPA alone. The treatment with NaPA alone at a dose of 6.9 mg/kg therefore appears to be nonsignificant, contrary to that with LS17-NaPAC at an equivalent dose of 60 mg/kg (p<0.005).

### EXAMPLE 21

### Study In Vitro of the Antiangiogenic Activity of LS17-NaPAC, of LS17-DMCB Alone and of NaPA Alone at Equivalent Concentrations (HUVEC Cells)

It has been previously shown that NaPA, on the one hand, and a functionalized dextran (CMDB7), on the other hand, have antiangiogenic, anti-invasive and antimetastic activities in vitro and in vivo (Adam et al., Cancer Re., 1997, 57, 1023-1029; Bagheri et al., Cancer Res. 1999, 59, 507-510).

Immortalized healthy endothelial cells (HUVEC) were seeded in a proportion of 10000 cells per well and per 100 μl of DMEM medium supplemented with 2% of FCS, in 96-well microplates (COSTAR®). The number of cells was determined after having prepared a standard range.

After culturing for 24 hours, the HUVEC cells were incubated with the various concentrations of the products to be tested prepared in DMEM medium containing 2% of FCS. The absorbance at the time the products were added was measured and compared to the standard range, so as to determine the number of cells.

After culturing for 72 hours, the cell supernatant is removed and 1 wash with PBS buffer containing calcium and magnesium is carried out. 100 μl of a solution of MTT is then added and the cells are incubated for 4 hours. After 4 hours of incubation, the MTT is removed and the crystals which have formed are dissolved with DMSO for 10 minutes with agitation. Absorbance is measured at 570 nm.

The concentration of 10 μg/ml of LS17-NaPAC tested is compared to a concentration of NaPA alone of 1.3216 μg/ml or to a concentration of 8.7 μg/ml of LS17-DMCB alone.

These results show that NaPA and LS17-DMCB tested alone do not appear to exhibit an inhibitory effect on HUVEC cell proliferation.

On the other hand, the LS17-NaPAC inhibits the growth of these cells in a dose-dependent manner after 72 hours of treatment. 50% inhibition is observed for a concentration of the order of 605 μg/ml.

Consequently, the LS17-NaPAC inhibits the proliferation of HUVEC endothelial cells. It may therefore be deduced therefrom that the LS17-NaPAC can be used to inhibit the proliferation of endothelial cells in order to decrease tumor vascularization (antiangiogenic activity).

1. A pharmaceutical composition, characterized in that it comprises, as active principle, at least one composition chosen from the group consisting of:
a) a conjugate of a polymer comprising at least one free hydroxyl function and of at least one phenylalkylcarboxylic acid derivative of formula (I) below:
R₂—(CH₂)ₙ—COOR₁ (I)
in which:
R₁ represents a hydrogen atom, a halogen atom, an atom of a monovalent alkali metal such as sodium or potassium, or a group —CO(CH₂)ₘR₃,
R₂ and R₃ represent an unsubstituted phenyl radical,
n and m, which are identical, are integers of between 1 and 3 inclusive;
said conjugate corresponding to formula (III) below:
(Polymer)-(OOC—(CH₂)ₙ—R₂)_{d} (III)
in which the polymer comprises at least one free hydroxyl function, n is an integer of between 1 and 3 inclusive, R₂ represents an unsubstituted phenyl radical and d, which is the index of substitution (ds) of derivative of formula (I), is ≧0.05 and ≦1.5, and preferably >0.15;
b) a combination of at least one polymer having a molecular mass of greater than or equal to 5000 Da and comprising at least one free hydroxyl function, and of at least one phenylalkylcarboxylic acid salt of formula (I′) below:
R′₂—(CH₂)_{n′}—COOR′₁ (I′)
in which:
R′₁ is an atom of a monovalent alkali metal such as sodium or potassium,
R′₂ represents an unsubstituted phenyl radical,
n′ is an integer of between 1 and 3 inclusive;
c) and mixtures thereof, and optionally one or more other additional active principles, and possibly in the presence or not of a pharmaceutically acceptable vehicle and/or of a physiologically acceptable carrier.

2. The pharmaceutical composition as claimed in claim 1, characterized in that the polymers are natural or synthetic and optionally functionalized, at the free hydroxyl functions, with one or more functional chemical groups chosen from carboxylic, alkylcarboxylic, arylalkylcarboxylic, N-benzylethylenecarboxamide, sulfate and sulfonate groups.

3. The pharmaceutical composition as claimed in claim 1 or 2, characterized in that the polymers are chosen from polyols and polysaccharides.

4. The pharmaceutical composition as claimed in claim 3, characterized in that the polysaccharides are chosen from glucosans such as starch, glycogen, celluloses, dextrans, poly-β-1,3-glucans and chitin; arabans; xylans and pectins.

5. The pharmaceutical composition as claimed in claim 4, characterized in that the functionalized synthetic dextrans are chosen from the dextran derivatives of formula (II) below:
DMCₐB_{b}Su_{c} (II)
in which:
D represents a polysaccharide chain, preferably consisting of sequences of glucoside units,
MC represents methylcarboxylic groups,
B represents N-benzylmethylenecarboxamide groups,
Su represents sulfate groups (sulfatation of the free hydroxyl functions borne by the glucoside units),
a, b and c represent the degree of substitution (ds), respectively of the groups MC, B and Su; a being equal to 0 or ≦2; b being equal to 0 or ≦1; c being equal to 0 or ≦1 and; it being understood that the sum of a+b+c≦3.

6. The pharmaceutical composition as claimed in claim 5, characterized in that the dextrans of formula (II) are chosen from those in which 0.5≦a≧1.5; 0.1≦b≧1; and 0≦c≧0.6.

7. The pharmaceutical composition as claimed in any one of the preceding claims, characterized in that the phenylalkylcarboxylic acid salts of formula (I′) are chosen from sodium phenylacetate, potassium phenylacetate, sodium phenylpropionate, potassium phenylpropionate, sodium phenylbutyrate and potassium phenylbutyrate.

8. The pharmaceutical composition as claimed in any one of the preceding claims, characterized in that the ratio of concentration between the polymer(s), firstly, and the phenylalkylcarboxylic acid salt(s) of formula (I′), secondly, is between 10:1 and 1:1.

9. The pharmaceutical composition as claimed in any one of the preceding claims, characterized in that it contains at least one conjugate of formula (IIIa) below:
(DMCₐB_{b}Su_{c}).(OOC—(CH₂)ₙ—R₂)_{d} (IIIa)
in which D; MC; C; B; Su; R₂; a, b, and c have the same meanings as those given in claims 1 and 5 and d has the same meaning as that given in claim 1, the sum a+b+c+d being less than or equal to 3.

10. The pharmaceutical composition as claimed in claim 9, characterized in that the conjugate compounds of formula (IIIa) are chosen from those in which a>0.5; b>0.15; c=0 or <0.8 and d<1.5.

11. The composition as claimed in any one of the preceding claims, characterized in that it has the following biological activities: antiproliferative, cytostatic, necrotizing, proapoptotic, antiangiogenic, antimetastatic and mitogenic factor-inhibiting activities.

12. The composition as claimed in any one of the preceding claims, characterized in that it is intended to be administered systemically.

13. The composition as claimed in claim 12, characterized in that it is intended to be administered at doses ranging from 0.1 mg/kg per day to 200 mg/kg per day, at a rate of twice a week.

14. A conjugate compound of formula (III) below:
(Polymer)-(OOC—(CH₂)ₙ—R₂)_{d} (III)
in which the natural or synthetic and optionally functionalized polymer comprises at least one free hydroxyl function, n, R₂ and d have the same meanings as those given in claim 1.

15. The conjugate compound as claimed in claim 14, characterized in that it corresponds to formula (IIIa) below:
(DMCₐB_{b}Su_{c}).(OOC—(CH₂)ₙ—R₂)_{d} (IIIa)
in which D; MC; C; B; Su; R₂; a, b, and c have the same meanings as those given in claim 9 and d has the same meaning as that given in claim 1, the sum a+b+c+d being less than or equal to 3.

16. The conjugate compound as claimed in claim 15, characterized in that it is chosen from those in which a>0.5; ; b>0.15; c=0 or <0.8 and d<1.5.
